(19) United States
(12) Patent Application Publication
(10) Pub. No.: US 2024/0032853 A1
(43) Pub. Date: Feb. 1, 2024

(54) Method and Apparatus for Detecting Conditions from Physiology Data

(71) Applicant: PhysIQ Inc., Naperville, IL (US)

(72) Inventors: Jadranka Sekaric, Naperville, IL (US); Stephan Wegerich, Geneva, IL (US); Steve Steinhubl, Anchorage, AK (US)

(21) Appl. No.: 18/226,408

(22) Filed: Jul. 26, 2023

Related U.S. Application Data
(60) Provisional application No. 63/392,218, filed on Jul. 26, 2022.

(51) Int. Cl. A61B 5/00; A61B 5/0205; A61B 5/11; G16H 40/67

(52) U.S. Cl. CPC A61B 5/41; A61B 5/4848; A61B 5/6801; A61B 5/0205; A61B 5/1118; A61B 5/7264; G16H 40/67; A61B 5/02405

(57) ABSTRACT

A computerized system for measuring and/or detecting responses or conditions in human beings based on data from wearable sensors worn in a natural free-living context. Based upon the measurements and/or detection, various actions can be taken.

### CROSS REFERENCE TO RELATED APPLICATION

This application claims the benefit of priority of U.S. Provisional Application 63/392,218, filed Jul. 26, 2022, which is incorporated by reference herein in its entirety.

### TECHNICAL FIELD

This invention relates generally to detecting inflammatory processes in humans and taking actions based upon these detections.

### BACKGROUND

Inflammatory responses occur in humans and can be thought of as a biological response of the immune system in a human characterized by the initiation of signaling pathways. The inflammatory response can be triggered by a variety of factors, including pathogens, damaged cells, toxic compounds, vaccines, and other immune system activators. In some cases, the inflammatory response can be considered to be due to adverse conditions (e.g., as the result of disease), but in other instances an inflammatory response can be considered as a positive (e.g., a positive result of receiving a vaccine).

### BRIEF DESCRIPTION OF THE DRAWINGS

FIG. 1 comprises a block diagram of a system for detecting inflammatory processes in humans and taking actions based upon these detections according to various embodiments of the present invention;

FIG. 2 comprises a flowchart of an approach for detecting inflammatory processes in humans and taking actions based upon these detections according to various embodiments of the present invention;

FIG. 3 comprises a flowchart of a general approach for using residuals to create an inflammation marker according to various embodiments of the present invention;

FIG. 4 comprises a flowchart of an approach for detecting inflammatory processes in humans and taking actions based upon these detections according to various embodiments of the present invention; and

FIG. 5 is an illustration of a safety monitoring system of the invention for use in remote monitoring of the patient in the at-home context.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required.

### DETAILED DESCRIPTION

The approaches described herein provide a computerized system for measuring and/or detecting adverse conditions or side effects (e.g., inflammatory responses) in a human being based on data from wearable sensors worn in a natural free-living context. Based upon the measurements and/or detection, various actions can be taken. By “inflammation” or “inflammatory responses,” it is meant as a biological response of the immune system characterized by the initiation of signaling pathways that can be triggered by a variety of factors, including pathogens, damaged cells, toxic compounds, vaccines, and other immune system activators. It will also be appreciated that these approaches can be utilized to detect positive effects on humans such as the efficacy of vaccines. Put another way, the approaches provided herein can be used to determine inflammation-related physiology changes in humans, whether that change is characterized as being positive or negative.

Measuring a side effect or condition such as an inflammatory response in humans is useful for a number of different reasons. To mention a few examples, measuring an inflammatory response is useful when safety monitoring (e.g., in the context of clinical care or a clinical trial); where a patient or participant may be sent home having had a treatment administered; and/or where side effects that manifest through activation of the immune system must be recognized and guarded against. Measuring an inflammatory response is also useful in the detection of reactogenicity of a vaccine, in order to register the efficacy or level of action thereof (e.g., immunogenicity) and safety across a representative population. Measuring an inflammatory response is furthermore useful when ascertaining whether a medical intervention is having a desired response, or if a treatment should be up-titrated or a booster round of a treatment added for a patient in whom insufficient response was seen.

Measuring an adverse or positive side effect or condition such as an inflammatory response is additionally useful in monitoring a patient in the home setting who is vulnerable to infection, such as sepsis or infection at the site of a recent surgery. Measuring an inflammatory response is also useful in identifying the overall course of inflammatory conditions such as autoimmune diseases like rheumatoid arthritis and an individual's response to therapeutic interventions.

In the approaches provided herein, sensors are utilized to obtain data from a human. In aspects, sensor data from the sensors comprises continuous vital signs data, such as a heart rate, respiration rate, core temperature, skin temperature, and activity, and statistical derivatives thereof, which are measured from wearable sensors throughout activities of daily living. Sensor data is collected and sent to a computerized platform, for example, in the cloud, where it is synthesized into an analysis looking at the inflammatory response of the patient.

The approaches provided herein generate a personalized (also referred to as “individualized”) baseline model of tandem vital sign behavior, typically based on one to several days of vital signs collected from the individual. By “tandem vital sign behavior” it is meant the characteristics of the interplay or cross-correlation between multiple vital signs over time and in response to exertion through activities of daily living. By using a baseline model, natural variation between individuals in the population can be controlled for, which might otherwise confound detection of the signal for an inflammatory response. The baseline model represents the way vital signs of the individual vary in tandem due to unconstrained activities of daily living. In one embodiment, the model is an auto-associative model or autoencoder that is constrained to recognize the coupling patterns of tandem vital sign variation in the individual and make estimates of expected vital sign behavior, as described in greater detail herein.

In many of these embodiments, physiology data is collected from at least one wearable sensor worn by the patient during a pre-treatment interval. An individualized estimator model is created based on the patient's pre-treatment collected physiology data. The model is capable of estimating expected behavior of physiological variables responsive to receiving new physiology data from the at least one wearable sensor worn by the patient.

Additional physiology data is collected from the at least one wearable sensor worn by the patient during a post-treatment interval. Estimates of the expected values of the post-treatment physiology data are generated using the individualized estimator model. Post-treatment physiology data is compared to the estimates thereof and a determination is made as to when a pre-defined effect pattern is present based at least in part on the comparison. When the predefined effect pattern is present, and action is determined and performed. The action is one or more of: triggering an electronic questionnaire to be prompted to the patient; providing instructions to the patient to take a measurement; providing instructions to the patient to contact the patient's clinician; triggering a ticket in a call center system to queue a call to the patient; creating a prompt in an app on the patient's phone to contact a clinician; providing instructions to the patient related to triaging the effects; transmitting a control signal to control medical equipment associated with treating the patient; transmitting instructions to a vaccine manufacture to alter a composition and/or dosage of a vaccine; and transmitting instructions to a treating clinician to alter a composition and/or dosage of a treatment. Other examples of actions are possible.

In some examples, the effects are adverse side effects. In other examples, the effects are positive such as being signs of efficacy of a vaccine.

In still other examples, the physiology data comprises heart rate data, respiration rate data, core temperature data, skin temperature data, and activity data. Other examples are possible.

In other aspects, the individualized estimator model is trained using data collected from the patient while in a free-living physiological state where an inflammatory status of the patient is stable and not expected to be changing. In yet other aspects, the comparing is performed by determining residuals between the estimates and the physiology data. In some aspects, the residuals are synthesized into a singular score, the score being a scalar index.

In still other aspects, the individualized estimator model comprises a neural network or system of neural networks. Other examples of models and modeling approaches are possible.

In others of these embodiments, a system for monitoring a patient for effects of a pharmacological therapy comprises at least one wearable sensor and a control circuit. The at least one wearable sensor is worn by a patient during a pre-treatment interval and is configured to collect physiology data from the patient.

The control circuit is coupled to the at least one wearable sensor, the control circuit configured to create an individualized estimator model based on the patient's pre-treatment collected physiology data, the model capable of estimating physiological variables responsive to receiving new physiology data from the at least one wearable sensor worn by the patient. The at least one wearable sensor collects additional physiology data from the at least one wearable sensor worn by the patient during a post-treatment interval.

The control circuit is further configured to: generate estimates of the post-treatment physiology data using the individualized estimator model; compare post-treatment physiology data measured by the sensor to the estimates thereof and determine when a pre-defined effect pattern is present based at least in part on the comparison; and when the predefined effect pattern is present, determine and perform an action. The action being one or more of: triggering an electronic questionnaire to be prompted to the patient; providing instructions to the patient to take a measurement; providing instructions to the patient to contact the patient's clinician; triggering a ticket in a call center system to queue a call to the patient; creating a prompt in an app on the patient's phone to contact a clinician; provide instructions to the patient related to triaging the effects; transmit a control signal to control medical equipment associated with treating the patient; and transmit instructions to a vaccine manufacture to alter a composition and/or dosage of a vaccine. Other examples of actions are possible.

In other aspects, the baseline model is trained using data collected from the individual while in a free-living physiological state where their inflammatory status is stable and not expected to be changing. For safety monitoring in a clinical trial or during clinical care, the training data is collected prior to administration of a drug or other therapeutic intervention with concerns of an inflammatory response. For detecting vaccine action, the training data is collected prior to receiving the vaccine. For infection detection, the training data is collected preferably before surgery, or at least at a time immediately post-discharge when an infection is not yet likely to have evolved to a systemic level of response. In the setting of autoimmune conditions, the training data is collected prior to the initiation of a new therapy that can decrease systemic inflammation.

In aspects, at least one diurnal cycle is utilized for training or personalizing the model, and preferably several days of data. The model switches over from being used in a learning (or training) mode to being utilized in a monitoring mode, after learning or personalizing from the initial data, and then can generate estimates of vital signs in response to receiving new data from the sensors. The estimates of vital signs are then compared to the actual measurements of vital signs to produce residuals, which is the estimate subtracted from the measured value. The residual can either be negative or positive depending on whether the estimate is higher or lower than the measured value. In addition, the residuals can be synthesized into a singular score, a scalar index of the change in the behavior vital signs compared to the behavior during baseline modeling. In aspects, such a score can be a number between 0 and 1, where zero represents that the vital sign behavior is similar to or the same as what was learned initially, and one would represent that the behavior of the vital signs has definitely changed from what was trained.

In other aspects, the approaches provided herein rely on the pattern or signature of residuals that is exemplary of an inflammatory response. Several patterns can be tested at the same time in the alternate to look for different ways in which the inflammatory response can manifest itself in the signatures. At the same time, other conditions must be met by all signatures. Conversely, signatures can be defined that are indicative of a reduction in inflammatory response. This is an application in which an individual is already inflamed during the baseline period. In other aspects, the approaches provided herein identify the persistence of a signature or any one of a set of signatures, over a window of time that is characteristic of an inflammatory response. Single transient values of the residuals or of any pattern or signature may not represent a genuine physiological response if it does not persist.

The analysis described with respect to the present approaches can be aggregated from many individuals to help inform drug or vaccine development, or can be presented to clinicians, the patient, and/or other clinical decision-makers for use in acting on the patient's condition. Such an action can be an outreach to the patient who may be remotely located from a clinical setting, in order to ascertain if the patient can confirm symptoms. In other examples, the action can be the automated sending of a survey to the patient via a smartphone provided to the patient as part of the system in order to have the patient answer questions confirmatory of or denying the symptoms. In still other examples, the action can be making or having the patient make other measurements with manual used instruments to provide more data confirming or not confirming the suspicion of inflammation. In yet other examples, the action is administering or having the patient self-administer interventions that remediate the source of the inflammation whether an infective agent or an autoimmune response, e.g., taking antibiotics or taking drugs that counteract the immune response, as appropriate. In still other examples, the action can be to control a call center ticket queuing system to create a new ticket, where a call center is staffed with trained clinicians who can contact the patient. Given the difficulty of finding a time for a patient and clinicians to talk, yet another example includes activating or revealing a button on an app on the smartphone of the patient, which enables the patient to automatically initiate a call to clinicians at a call center trained to handle the patient's condition, which the patient can click on when the patient is in a situation permitting a discussion.

Wearable sensors can include any device that is capable of registering signals representative of heart rate, temperature, activity, respiration rate, and other behavioral and physiologic parameters. Statistical derivatives of these values, such as heart rate variability, may also be used in generating the baseline model. An exemplary sensor comprises a chest worn adhesive patch that is capable of collecting continuous electrocardiogram (ECG) signals, accelerometry and temperature(s).

In general, there is clinical interest and value in monitoring patients for signs of acute immune system activation in populations at risk. In addition, in some individuals, there is value in detecting evidence of the offset of chronic inflammation in response to therapeutic interventions designed to diminish inflammation. In aspects, the present approaches are directed towards situations in which it is important to detect changes in systemic inflammation that can be life-threatening (e.g., cytokine release syndrome or infections) or can be useful to optimize efficacy and safety of a therapy (e.g., vaccines or biological medical products), which can occur at any time, occurs systematically, and can occur at a wide range of severities, and for which it is necessary or beneficial to provide continuous surveillance. In this sense, “acute” means over a time period measured in hours to weeks, as opposed to long-term inflammatory processes which are measured in months to years.

To take one of these examples, immunotherapy is a treatment for cancer whereby the patient's own immune system is recruited to attack the cancer. An example of immunotherapy is CAR T-cell therapy, which involves re-engineering a person's own T-cells to have special receptors called chimeric antigen receptors (CARs). Such receptors help the T-cells to recognize and attack cancer cells.

More specifically, in a first step, the patient donates blood to get the T-cells. In a second step, in a laboratory, the T-cells are genetically engineered to encode a tumor-specific CAR. In a third step, the CAR T-cells multiply. In a fourth step, tests ensure safety and purity of manufactured CAR T-cells, which are then cryopreserved. In a fifth step, CAR T-cells are infused into the patient. In a sixth step, CAR T-cells hunt for cancer cells, bind to them and destroy them.

A side effect of immunotherapy for cancer is cytokine release syndrome. Cytokines are small proteins that are involved in messaging to orchestrate immune response in the body. In cytokine release syndrome, however, there is a flood of cytokines released which can be harmful to organs and in severe cases cause death. Cytokine Release Syndrome (CRS) may develop in a time frame of about 3 to 14 days after administration of the immunotherapy. Because of the potential risk, therapy has been administered in a clinical setting and the patient has been kept in the hospital for the entire time. This makes treatment very expensive. Advantageously, the approaches provided herein allow the patient to be sent home with a surveillance system that detects early signs of CRS so that clinicians could jump on it if it is detected. This reduces costs as compared to previous systems.

Typical known symptoms of CRS include fever, drop in blood pressure, and drop in SpO2, at various stages of the acute phase. An increase in heart rate may attend early stages; changes may also occur in heart rate variability and respiration.

Using the approaches provided herein and prior to administration of the CAR T-cells (the fifth step described above), a patient is provided a wearable sensor kit to wear sensor(s) at home for ˜1-4 weeks, 24 hours per day. This can also occur before the first step mentioned above, and the timing may depend on other steps being taken to treat the cancer or prep the treatment.

Data during this period prior to step 5 above are used to train a personalized baseline model. The patient is then brought into a clinic to receive treatment. CAR T-cells are infused to patient. The patient is sent home with wearable sensors and possibly with other manual measurement devices like a blood pressure cuff. Data is monitored from the patient and assessed according to the approaches provided herein. The system sweeps the incoming data for one or more signatures of CRS or general inflammatory response, as described in more detail below.

If a detection is made, this is provided to the clinicians via one of several notification methods, including sending an alert on web-enabled clinical portal. Push alerts can be sent to other medical monitoring systems. Push-alerts may additionally be sent as text messages or to the email address of clinicians. Push-notification to a smartphone may be provided to the patient, with instructions to contact their clinical care provider; or to click on a newly generated button in the app to automatically connect to a clinician call center. Push-notification can be sent to the patient via a smartphone, asking the patient to answer survey question(s) about status, which is then pushed or sent to the clinician. Push-notification can be sent to the patient on a smartphone, asking the patient to take a manual temperature reading or a manual SpO2 reading or a manual blood pressure reading and enter it to a smartphone application (app), which is then pushed or sent to the clinician.

Another potential use of the approaches provided herein involves vaccinations. Vaccination activates the innate immune system, triggering the synthesis of inflammatory cytokines critical to launching an antigen-specific adaptive immune response. The physical manifestations of this inflammation, termed reactogenicity, have historically been tracked only by symptom surveys. Limited studies directly measuring inflammatory blood biomarkers have not only found substantial inter-individual variation in this inflammatory response, but also a strong correlation between this response and both systemic symptoms and humoral immune response. Due to the lack of any scalable method of measuring an individual's response to a vaccine, for most people, the ultimate measure of their adequacy of immune protection from the vaccine is whether they experience a breakthrough infection and its severity.

In addition, objective evidence of individual inflammatory response to a vaccine can aid in the design of safer, better-tolerated vaccines. The limitations of the current gold-standard for safety tracking, subjective surveys, was highlighted in an analysis of reported adverse events in the placebo-controlled COVID-19 vaccine trials that found that over 50% of the systemic adverse events reported could be attributed to a “nocebo” response.

In a use case involving vaccines, the approaches provided herein include a surveillance system as described below. In aspects, individuals would initiate monitoring prior to receiving their vaccine and continue for 5-10 days after.

In clinical trials of vaccine development, the clinical trials could incorporate wearable sensors to better evaluate in larger populations the variability in the inflammatory response to various vaccine doses in different age groups to inform optimal dosing strategies that optimize efficacy and safety. Variability of each study participant in a study cohort can be assessed by monitoring prior to vaccine administration, to build a multi-variate baseline in the context of activities of daily living. Vaccine would be administered. Ongoing monitoring would occur for the period spanning an expected response. The degree of individual inflammatory response is assessed by methods described below, comparing the baseline to the post-vaccination period. The study results can be statistically analyzed.

For identifying an individual's unique response after receiving a vaccine, many known and unknown factors can influence a person's response to a vaccine. Known factors include age, sex, and co-morbidities, especially conditions/treatments affecting the immune system. Individuals in whom an expected inflammatory response is not identified can undergo further testing (e.g., blood levels of vaccine-induced antibodies) or be scheduled for a booster dose. For some individuals who do not experience any symptoms after vaccination, appropriate changes as detected by the surveillance system can reassure them.

Another example of situations where the present approaches can be deployed involves the early detection of sepsis or other infection manifestations. The primary function of the immune system is to defend the body from viral and bacterial pathogens. This response results in inflammation, which when severe enough manifests as a fever. However, a fever (e.g., temperature>38° C.) is a lagging indicator of infection as it is based on a population metric and is only a single measure of inflammation-induced changes.

For many individuals, such as those who are immunocompromised, the earliest possible detection of infection can be lifesaving. For others, early detection can prevent re-hospitalization and significant morbidities. For individuals at heightened risk, for example after surgeries, especially transplantation, active surveillance for the earliest signs of immune system activation and inflammation can potentially trigger early testing with blood cultures and/or more intensive monitoring in a healthcare facility.

In this use case, a baseline of multivariate vital sign behavior is built from monitored data from unconstrained activities of daily living, when it is known the patient does not have sepsis. Subsequent monitoring with the same sensors over a window of time where risk of sepsis is of concern then uses the baseline model to provide a comparison to monitored data, such that the signature of the early signs of inflammation attendant to sepsis are revealed when comparing to the multivariate baseline as described below. When alerted, clinical personnel can then intervene with appropriate steps to prevent a full-blown case of sepsis from developing.

Aspects of the operation and implementations of the present approaches are now described. Inflammatory processes affect measurable vital signs in a signature fashion. For example, increases in temperature, increases in heart rate (HR), increases in respiration rate (RR), and decreases in heart rate variability (HRV) may be the results of inflammation.

While long-window averages may be used to detect inflammation post hoc, it is advantageous to detect the inflammatory process on the time scale that it can urgently unfold, namely hours. In that time frame, long-window averages are not very useful. The challenge is that relative changes in one or more of the above must be detected (a) for an individual and (b) against a backdrop of normal variation due to free-living activities and behaviors.

The approaches provided herein establish a personalized multivariate dynamic baseline for detecting relative abnormalities from normal dynamic behavior of these vital signs. These approaches then measure whether the residuals are positive or negative, and in this way provide evidence of the above signature. For example, it may be determined whether the measured HR or RR higher than it is supposed to be; whether temperature higher than it is supposed to be; or whether HRV lower than it should be.

In aspects, a priority of these changes is determined and observed. In some examples, HRV is the most significant factor, and this change is believed in some aspects to be the earliest evidence having inflammation specificity. Temperature rise is slightly lagging, respiration rate is subject to some degree of voluntary control and is harder to reliably measure, and heart rate can be anomalously high with less specificity for inflammation (i.e., other reasons may cause this). Hence, the physiology of these four factors, and in particular HRV, are utilized. While evidence may be provided by all of these vital signs, some of these approaches focus on at least detecting a relative drop in HRV as estimated by a multivariate model.

Vital signs used by the present approaches include those that characterize the performance of the cardiopulmonary system. Several vital signs can be used as described below.

Heart rate (FIR) can be used. HR is typically expressed as the count of heart beats per minute, but which can be determined on a beat-to-beat basis by measuring the time interval between QRS peaks of the ECG, and then converted to an average HR over a time window such as a minute, to produce a minute rate. A minute-average heart rate can be determined as a trim mean of the inter-beat-intervals over a minute, then inverted to provide beats per minute. This can also be done from a PPG waveform as well or a ballistocardiograph, to mention other examples.

Respiration rate (RR) can also be used. RR can be measured from a plurality of sources including motion from the chest wall as measured by accelerometers in an adhesive patch; by respiratory sinus arrhythmia which represents the slight slowing and speeding-up of the heart rate with each breath; or by measuring the envelope of the ECG QRS peaks which vary as the chest compartment changes gas volume.

Gross activity (ACT) can also be used. Gross ACT can be measured as the standard deviation of the vector magnitude of the accelerometer deviations. However, other quantifications of movement are usable, such as “activity counts,” which is a long-used method of quantifying motion of a vibration sensor.

Heart rate variability (HRV) can also be utilized. HRV can be measured in a number of ways using the beat to be heart rate over a window of time. HRV may involve a standard deviation of “Normal” R-R intervals (SDNN); standard deviation of all R-R intervals (SDRR), standard deviation of the average NN intervals for each 5-minute period over a 24-hour window (SDANN); the root mean square of successive R-R interval differences (RMSSD); and/or the spectral HRV frequency bands; or Poincare HRV dispersion parameters.

The skin temperature (TEMPS), which is measured typically by a skin facing thermistor that is a part of the sensor device can also be used. In addition, the core body temperature (TEMPc) can be used. TEMPc can be predicted on the basis of two thermistors on a sensor, one of which is skin facing and the other of which is pointing to ambient air on the outer side of the sensor. Core temperature is then a function of heat flux through one side and out the other of a sensor that has both thermistors.

ECG waveform changes can be used. For example, the QT interval is measured as the time difference between the start or peak of the QRS complex of an ECG and the end of the T wave on the ECG or can be measured by a proxy such as the time interval to the peak of the T wave.

Since multivariate modeling of relationships among vital signs is facilitated by contemporaneous “snapshots,” it is best to provide windowed statistics (average heart rate, maximal activity, 95^{th} percentile temperature, by way of example) of the vital signs, over common windows that are assigned the same time stamp. In other words, each input to the downstream modeling will comprise a vector comprising a datum for each vital sign at the point in time represented by the vector. An example would be a vector of one-minute averages for each of HR, HRV, RR, ACT, TEMPc, and QT.

As mentioned elsewhere herein, the present approaches use personalized estimator models. In aspects, the personalized estimator models are any multivariate model that provides as an output an estimate of the expected value(s) of one or more vital sign values measured from the patient. Such models can be generated by Decision Tree/Random Forest function approximators; Similarity-based Models, and Neural Networks to mention a few examples.

In aspects, training data is required, which is captured from the patient prior to monitoring for adverse events, e.g., CRS or sepsis. Once training is complete, the personalized model is used to generate estimates responsive to input of newly measured data from the patient. Input comprises new readings of multivariate vital signs data from the wearable sensor. Output from the model comprises estimates of the input, which estimates are then used to generate residuals, namely the measured values minus the estimated values, as described below.

Similarity Based Modeling (SBM) is an auto-associative or autoencoding pattern reconstruction technique. At any point in time, a set of readings from all variables can be thought of as an input pattern (analogous to pixels in an image). The auto-associative estimation process is then a reconstruction of the input pattern based on the learned patterns used to generate the SBM model. SBM essentially attempts to reproduce an input pattern based on the information stored in the training data. The reproduction can only occur when a linear combination of the training data can be determined that fits the input pattern. This is possible when the input pattern is representative of the multivariate behavior in the training data. If the input pattern is not representative of the training data behavior, one or more of the estimated pattern elements will not closely match the corresponding input element. In effect, the estimates reflect what SBM believes each pattern element should be based on the model training data and the information present in the input pattern. The residual pattern (difference between the input and estimated pattern) highlights the locations within the pattern that deviate from the training data. These differences are often subtle and so are accumulated over time to drive decision processes.

The mathematics behind the SBM approach are centered on the application of a “similarity operation” on pairs of observation vectors and the manipulation of a “state” matrix D containing a set of historical training vectors (input patterns). The number of columns in D is equal to the number of representative training vectors (M) and the number of rows is equal to the number of data sources contained in each vector (L). Defining the set of measurements taken at a given time nⱼ as a training vector, x(nⱼ),

x(nⱼ)=[x₁(nⱼ)x₂(nⱼ)x₃(nⱼ) . . . x_{L}(nⱼ)]^{T} (1)

where xᵢ(nⱼ) is the measurement from a data source i at time nⱼ, then the state matrix D is given by:

D=[x(n₁)x(n₂)x(n₃) . . . x(n_{M})]. (2)

The result of the similarity operation for two observation vectors is a similarity score (a scalar). The similarity operation is nonlinear but can be extended to matrix operations, where a scalar similarity score is rendered for each combination of two vectors stored in two matrices of appropriate dimensions. Accordingly, given an input vector (or pattern) xᵢₙ containing single readings from each of the L data sources, a vector of corresponding estimated data source values, xₑₛₜ, is determined from (3) through (5). The estimates for each variable in the input form a “reconstructed” input pattern using a linear combination of the training vectors in D.

xₑₛₜ=D·w (3)

Here, w is a set of weighting factors derived from the following.

$$w = \frac{\hat{w}}{Σ \hat{w}} \qquad ( 4 )$$

$$\hat{w} = {( {D}^{T} ⊗ D )}^{- 1} \cdot ( {D}^{T} ⊗ {x}_{i n} ) = {G}^{- 1} \cdot a \qquad ( 5 )$$

The similarity operation is indicated by the symbol “⊗” not to be confused with the Kronecker product. Generally, the vectors stored in D are chosen in such a way that attempts to uniformly span the dynamic variation range of the monitored process. However, in many applications including human health monitoring, this may not always be possible without creating an extremely large D matrix since the number of feasible operating regimes is very large. In practice, a large D matrix generally results in an SBM model that over-fits the input data. The ability of SBM to do early anomaly detection is diminished when a model over-fits the monitored data. In this situation, the estimate follows or tracks a progressing anomaly instead of deviating from it over time. One way to address this issue is to generate several SBM models, each covering a subset of all operating regimes. Another way is to modify the SBM algorithm so that it is localized to the current operating regime at any point in time.

Localization is provided. The localized SBM approach hinges on generating a D matrix dynamically to characterize only the local behavior of a system at a point in time. The idea is to select a subset of data to define a state matrix D(t) that is most relevant to the current input pattern from a much larger superset matrix H that characterizes the full dynamic range of the monitored system. The estimate is then generated as shown in (6) through (8) based only on these selected, currently relevant, vectors. The process is repeated for each new input vector. The weighting factors thus become,

D(t)={H|F(H,xᵢₙ(t))} (6)

ŵ=(D(t)^{T}⊗D(t))⁻¹·(D(t)^{T}⊗xᵢₙ(t))=G(t)⁻¹·a (7)

where F(·,·) in (6) is a relevant vector selection process given an input vector xᵢₙ(t) and a reference data matrix H. Finally, the residual vector at time t is given by (10).

r(t)=xᵢₙ(t)−xₑₛₜ(t) (8)

Random forest approaches can also be used. A “Round Robin” configuration may behave like an auto encoder/auto-associative model, where for M variables we have M RFs each producing a single output and together represents M estimates for M inputs. Each RF is trained to be personalized to the patient using the data from the patient's pre-treatment data collection. Neural networks can be used to implement the personalized models provided herein. When trained with sufficient examples, neural networks can be highly effective in non-linear pattern recognition, non-linear encoding of key features from raw input patterns, and inferential function approximators. In practice, a challenge for use cases contemplated herein is that instrumenting a patient with sensors during a pre-treatment period may not yield enough raw vital sign data to effectively train a neural network from scratch to make personalized estimates of vital signs for the patient. Accordingly, in one approach according to the invention, a first neural network is developed to be an “encoding” neural network that accepts as input time series of a first tranche of one or multiple days of vital sign data from any person, and outputs a multi-element encoding vector to represent the personalized cardiopulmonary behavior of that person. Second, an ensemble of one or more estimator neural networks are developed to take as input the encoding vector, as well as a second tranche of vital sign data from the person (not overlapping with the first tranche) in order to make estimates of expected values for the second tranche of vital sign data. The ensemble comprises individual neural networks that each estimate one vital sign given input of all the other vital signs: e.g., one member of the ensemble estimates heart rate, given input of activity, HRV, respiration, temperature and the encoding vector, while a second member of the ensemble estimates HRV, given input of the activity, the heart rate, the respiration rate, temperature and the encoding vector, and so on. Each ensemble member thus makes an inferential estimate of the vital sign that is not present in its input.

These networks are pre-trained using vast amounts of vital sign data from at least dozens or hundreds of individuals, prior to being deployed as personalized estimators. Each training sample comprises a first tranche and a second tranche of vital sign data from a unique person in the training set. First tranche data is input to the encoding neural network, which produces an encoding vector. The encoding vector plus the second tranche data is input to the ensemble of estimator networks, which accordingly generate each of their respective vital sign estimates as outputs, which were not present as inputs, i.e., they were inferentially estimated. These inferential outputs are compared to actual known vital signs from the second tranche to determine an estimation error, which is backpropagated in a cost function to train both the ensemble of estimators as well as the encoding network. This is done over a large number of matched first and second tranches of data, so that the encoding network becomes effective at generating an encoding vector that accurately encodes the cardiopulmonary behavior of anyone whose data is input to the encoding network; and the estimator networks each become effective at making accurate inferential estimates of their respective inferred vital sign in a second tranche of data from a person, given an input of the encoding vector representing the person, and a second tranche of data from the person. Input to the encoding network can be structured for example as several days of one-minute vital sign data; input to the estimator networks can be structured as the combination of the encoding vector that results from the first network, and a window of time series vital sign data that allows for timely monitoring of inflammation response. For example, this window can be a 3-hour window; or a trailing 24-hour window with a slide of 3 hours. However, for clarity, the pre-training of these networks does not involve anyone who is undergoing an inflammatory response. Rather, pre-training involves data where the first tranche and the second tranche of data from a person can be expected to be representative of unchanged physiology. In other words, these networks are being trained to make accurate estimates of what second tranche data should be, given the encoding for the first tranche.

Once pre-trained, these neural networks become effective at making personalized estimates as follows. Pre-treatment data from a patient is input to the encoding network, to generate an encoding vector. This is the personalization step, as this encoding vector now tailors the estimator networks to make estimates of expected vital sign behavior specific to the patient. Thus, by way of illustration, when a window of post-treatment vital sign data excluding heart rate is input to that ensemble member that estimates heart rate, it will make estimates of what the expected heart rate should be, given the encoding vector from pre-treatment plus the other non-heart-rate vital signs from a window of time in post-treatment. That estimated heart rate can then be compared to the actual measured heart rate from the sensor, to create the aforementioned residual for heart rate. Other ensemble members can do the same for other vital signs. In this way, residuals for all vital signs needed for the detection or quantification of inflammatory response (as a change from normal health) can be provided by neural networks on a personalized basis.

Residuals are utilized by the approaches provided herein. Residuals are equal to each measured vital sign value minus the value of the corresponding estimate. For example, if an average one-minute Heart Rate is being used as a monitored parameter, the measured values of contemporaneous vital sign values are input to the model, and the model outputs a value for Heart Rate that is the anticipated or expected heart rate for the person given the other vital signs as an ensemble. Then, the residual for heart rate is the measured heart rate minus the estimated heart rate.

Residuals thus comprise time series in their own right, one for each vital sign in the model. The value and sign (positive or negative) of the residual has particular meaning.

For example, a high residual (positive, >0) means the measured value is higher than the model anticipated for normal physiology for that patient. If the residual is low (<0) then the measured value is below what was anticipated for normal physiology for the patient. Residuals can then be examined in the rules for detection described below.

In addition to individual residuals, a singe scalar index of overall derangement of vital sign behavior can be generated that combines the normalized magnitudes of the residuals at each measurement. One example of a single scalar index is the Multivariate Health Index described with respect to U.S. Pat. No. 8,620,591, which is incorporated herein by reference in its entirety, also known as a multivariate change index (MCI) as it will be referred to herein.

In summary, the personalized baseline model is used to generate estimates for at least a portion of the training data set, and the relative sizes of residuals are used to scale a distribution of the expected dispersion typical for residuals from the baseline under normal (non-deranged) vital sign behavior. As new data observations are input during monitoring mode, the resulting residuals can be compared to this distribution in a way that can generate a single score scaled to the range of 0 to 1, that relates to how likely it is that the vector of residuals is a member of that expected distribution or not. When the vector of residuals is well within the typical distribution (as characterized by the training data scaling results) then the output of the MCI is closer to 0 (low likelihood that vital signs are behaving differently) whereas if the vector of residuals is more in the tail of the multidimensional distribution, then MCI is closer to 1 (high likelihood of changed behavior). In this way, the MCI is a measure of the likelihood of deranged vital sign behavior, regardless of the underlying activities of the person (i.e., doesn't matter if they are sleeping, watching TV, running, etc.).

The MCI is thus a time series that has a data point for each input observation of vital signs that is estimated by the personalized model. The value of MCI itself can be used as part of the rule patterns provided by the approaches described herein.

Other methods for providing a single scalar index for the multidimensional vectors of residual data can be used as alternatives to the above-described MCI. For example, Euclidean distance is a well-known way of measuring the distance of a multi-dimensional point from a distribution of points and can be used as follows: (a) residuals from training samples (no inflammation) are normalized to a mean of zero and standard deviation of 1; (b) the “center” in multi-dimensional space of the residuals from training samples is found; (c) a range of distances from that center is defined such that maximum likelihood of normalcy (set to 1) is at zero distance and a minimal likelihood of normalcy (set to 0) is defined at some maximum distance equal to a multiple of the standard deviation (higher distances are clipped at the maximum); (d) each residual vector for a test observation is normalized and its distance computed to map into the range 0 to 1 of likelihood of normalcy. Euclidean distance assumes a spherical distribution; other techniques such as the Mahalanobis Distance, are potentially better with correlated variables like the vital signs used herein. The principle is the same, however, in mapping multivariate residual vectors from tested data to a distribution expected for normal training data, to provide a likelihood that can be mapped into a range of zero to one that the residual vector is abnormal or not and by how much, just like the MCI technique. All such methods of producing a single scalar index of change are contemplated as signals usable in the rules of the present invention, when applied to the residuals from personalized modeling for expected values. It should be understood in this disclosure that where “MCI” is used herein for detection or quantification of inflammatory response, other single scalar indexes can be substituted in the alternative according to the invention.

The concept of rule patterns (e.g., implemented as computer code) for the detection of inflammation according to the approaches provided herein is that there is a signature of inflammation in the residuals as a time series. As discussed herein, the hallmark pattern involves one or more relative changes given the personalized physiology and the free-living context of the person's behavior. For example, HRV that is lower than expected, HR that is higher than expected, temperature that is higher than expected, and/or respiration rate that is higher than expected may all be such relative changes.

For example, during inflammation, it is reasonable to assume that the heart rate increases above what it would normally be expected. The personalized model provides the ability to see this elevation in heart rate above normal rates by masking out heart rate raw value changes that are due to normal activities of daily living, and the elevation is seen as a positive residual value. The same can be true for respiration rate.

In addition, the rule can be gated by the magnitude of the MCI, which quantifies an overall degree of derangement of the system of vital signs in tandem. When measuring vital signs in a free-living context, it may be difficult for a personalized model to isolate change to one or another vital sign—the model may suffer “spillover” where deviation in one variable is imbued to the estimate of another variable and the latter variable appears to be anomalous. Trivial or small deviations of the above vital signs may not rise to a reliable signal of inflammation; by adding in a requirement for at least a minimum amount of overall derangement among the vital signs, the MCI ensures a more reliable detection.

A rule for detecting a pattern may also have non-residual signals in it, e.g., that the core temperature is simply higher than a threshold, as human body temperature is normally held within tight bounds for homeostasis regardless of activities of daily living (granted that it declines while sleeping, and rises during exercise, but these changes are relatively small). Alternatively, the absolute value of the MCI can be tested as part of the rule.

In general, elements of a rule that detects any pattern generically can be chosen from several factors. These factors include the directionality of the residual, e.g., positive or negative; the magnitude of residual; magnitude of MCI; the duration or persistence of pattern; and/or any filters applied to absolute value vital signs or conditions to mention a few examples.

The output of the rule can be a modification of a time series, such as the MCI, to modulate its value when the rule is true or false, or a trigger an alert when the rule is met. Other examples are possible.

In one example of the operation of a rule, an operative rule would check for a negative HRV residual (drop in HRV relative to expected value) in the presence of a sufficiently large MCI.

In another example of the operation of a rule, the rule would check to assure MCI is sufficiently high and HRV residual is not positive, and then given that gating condition, check for any of a positive HR residual, a positive RR residual, a positive temperature residual, or a negative HRV residual.

In still another example of the operation of a rule, the rule transforms the MCI value based on the rule being false (inflammation pattern not detected) to a zero value, e.g., MCI becomes inflammatory MCI (iMCI) such that its value is preserved if inflammation is recognized or zeroed out if inflammation is nor recognized. This makes MCI specific to inflammation, and “iMCI” is essentially a univariate marker for inflammation.

In yet another example of the operation of a rule, the rule processing then compares the iMCI to a threshold to trigger a detection alert if the iMCI exceeds the threshold.

In still another example of the operation of a rule, the rule uses a window of iMCI values to create an average value over a window, to smooth the signal, and uses the smoothed signal for quantification or for triggering detection.

In yet another example of the operation of a rule, the rule performs an additional transformation on the iMCI values where any highest values is “latched” on at that value until a next highest value is encountered, or a sufficiently lower value is encountered (e.g., keep the signal high until it drops lower by at least a certain amount indicating evidence of inflammation is decidedly less).

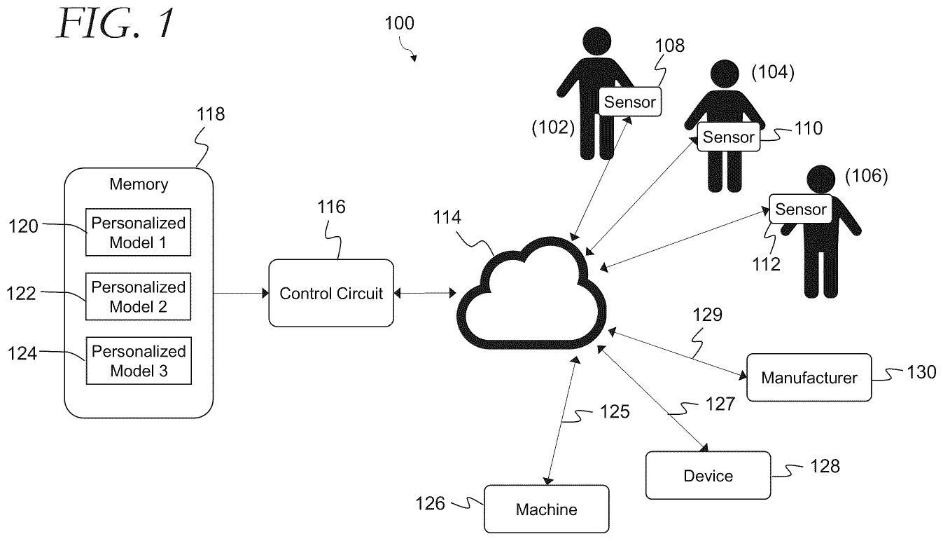

Referring now to FIG. 1, one example of a system 100 for determining and acting upon the inflammatory responses of humans is described. The system includes a first human 102, a second human 104, and a third human 106. A first sensor 108 is worn by the first human 102, a second sensor 110 is worn by the second human 104, and a third sensor 112 is worn by the third human 106. An electronic network 114 is coupled to the sensors 108, 110, and 112, and a control circuit 116.

A memory 118 is coupled to the control circuit 116. The memory 118 stores a first personalized estimator model 120, a second personalized estimator model 122, and a third personalized estimator model 124. The electronic network 114 is also coupled to a machine 126, an electronic device 128, and a manufacturer 130. Other devices or systems may be coupled to the network 116.

The first human 102, second human 104, and third human 106 are, in one example, patients. They may be treated for certain illnesses or may simply be interested in monitoring their health. In some examples, they may be participating in a clinical study.

A first sensor 108, second sensor 110, and third sensor 112 may be any type of sensor or sensor arrangement (including multiple sensors) that can be worn by the humans 102, 104, and 106. The sensors may be disposed at any convenient place on the human body such as the torso or wrist. In some aspects, a torso-adherent sensor patch is used and continuously worn by the humans that enables measurement of several values. These values may include the heart rate and HRV from a single lead ECG, movement quantification (activity) from a 3-axis accelerometer, respiration rate from derivatives of movement, or amplitude/frequency modulation of HR, and/or skin temperature at the sensor site. As mentioned above, the above-mentioned vital signs can be computed from the waveform data for ECG and 3-axis accelerometer. By way of example, the ECG may be sampled at 125 Hz, and the accelerometer sampling rate may be 15 Hz or greater. Other examples are possible.

The electronic network 114 is any type of electronic communication network or combination of networks such as a wireless network, the internet, a local area network, a wide area network, to mention a few examples.

A control circuit 116 is any type of processing device, processor, controller and includes, as examples, all types of electronic controllers, microcontrollers, servers, or microprocessors. The control circuit 116 may also include an internal memory that stores computer instructions that are executed to implement the functions, rules, or other operations described herein. The control circuit 116 and memory 118 may be disposed at a central processing device or central call center.

The memory 118 is any type of memory device or database or combination of devices. The memory 118 may be a read-only memory, random access memory, programable read only memory, or combinations of these and other types of electronic memories to mention a few examples.

The first personalized estimator model 120, second personalized estimator model 122, and a third personalized estimator model 124 are configured to estimate physiological variables responsive to receiving new physiology data from the sensors 108, 110, and 112 worn by the humans 102, 104 and 106. The personalized estimator models 120, 122, and 124 are any multivariate model that provides as an output an estimate of the expected value of one or more vital sign values, given the measured values from sensor(s) on the patient. Such models can be generated by Decision Tree/Random Forest function approximators; Similarity-based Models, and Neural Networks to mention a few examples.

The machine 126 is, in one example, a medical device utilized by one or more of the humans 102, 104, and 106. For example, the machine 126 may dispense medicine, monitor the human 102, 104, or 106, or perform some other function. An electronic control signal 125 may be sent by the control circuit 116 to the machine 126 to control the machine 126 and/or aspects of operation of the machine 126. The control signal 125 may activate the machine 126, deactivate the machine 126, control operating parameters of the machine 126 (e.g., the speed, amount of medicine dispensed, operations of a display at the screen on the machine 126, information displayed upon this display, or how often the machine is monitoring a human 102, 104, or 106 to mention a few examples).

The electronic device 128 may be a smartphone, laptop, personal computer, or cellular phone to mention a few examples. Electronic signals 127 may be sent by the control circuit 116 that may be electronic messages, control signals, or any other type of electronic signals. For example, the electronic device 128 may be used by one of the humans 102, 104, or 106 and be used to display alerts, instructions, or other information to the humans 102, 104, or 106. In other examples, the electronic device 128 may be utilized by medical personnel (e.g., doctors, nurses, hospitals, or therapists) that are treating the humans 102, 104, or 106. Although only one electronic device 128 is shown, it will be appreciated that multiple devices may exist and be operated by different individuals or institutions. In addition, the electronic device 128 may communicate with the control circuit 116, and through the control circuit 116, with other devices or systems coupled to the network 114. The electronic device 128 may itself include a processing device or control circuit to perform the operations described.

The manufacturer 130, in one example, is a vaccine manufacturer. The control circuit 116 may send control signals or other electronic instructions 129 to the manufacturer 130. These instructions 129 may automatically cause or inform the manufacturer 130 to alter a process. In one example, the manufacturer 120 is a vaccine manufacturer and the electronic instructions 129 cause the manufacturer 130 to alter the composition, dosage, or some other characteristic of a vaccine (or drug) being administered to one or more of the humans 102, 104, and 106. In some aspects, this may automatically control machines and/or processes (at a facility of the manufacturer 130) creating the vaccine or drug. In other examples, the electronic instructions include alerts or other messages to the manufacturer 130 with suggested or proposed changes to the vaccine or drug. The manufacturer 130 may utilize electronic receivers, transmitters, transceivers, memories, databases, servers, processors, control circuits, displays, computers, and/or other electronic devices (and combinations of these devices) to perform these functions.

During pretreatment of the humans 102, 104, and 106, training of the models 120, 122, and 124 occurs. When the models are neural networks, it will be appreciated that the physical structure of these neural networks are changed. For example, the weights, layers, or other structures of the neural network are changed from a first structure or state to a second structure or state. It will also be understood that the structures of the resultant trained models 120, 122, and 124 are unique as to each other. Each trained model 120, 122, and 124 is trained specifically only on data from a corresponding human. Trained model 120 has only been trained on data from human 102, trained model 122 has only been trained on data from human 104, and trained model 124 has only been trained on data from human 106. Thus, the models are completely different from each other, unique, personal to a unique human, and cannot be viewed as general purpose computing resources. Application of the same data to different models will not necessarily produce the same or similar results.

During a monitoring or execution (subsequent to the training phase), the control circuit 116 applies incoming data from a particular human 102, 104, and 106 to the model 120, 122, and 124 associated with the patient. In aspects, the control circuit 116 may select or retrieve from the memory 118 the proper model 120, 122, or 124 for particular data that is to be received and processed. In examples, the data may indicate the human for which the data originates, or a technician may electronically inform the control circuit 116 of the origin of certain data so that the control circuit 116 can select the correct model.

Application of the data to a model produces as estimate of the data. The estimate may be for one or more parameters such as heart rate, heart rate variability, respiration rate, activity, temperature, and so forth. The estimates are compared (on a parameter-by-parameter basis) to the actual data and a difference obtained. The difference is a residual and the residuals can be determined over time and their patterns analyzed by the control circuit 116 as described elsewhere herein. The results of the analysis can be used by the control circuit 116 to determine and perform actions. These actions can include, as mentioned, controlling the machine 126, sending instructions to the electronic device 128, and/or sending instructions to the manufacturer 130. It will be appreciated that only the machine 126, electric device 128, and manufacturer 130 are shown here but that other devices may be controlled or informed by the control circuit 116. It will also be understood that communications as between the control circuit 116 and these devices may also be two-way communications, that is, the devices may send instructions or other electronic information to the control circuit 116 for other purposes.

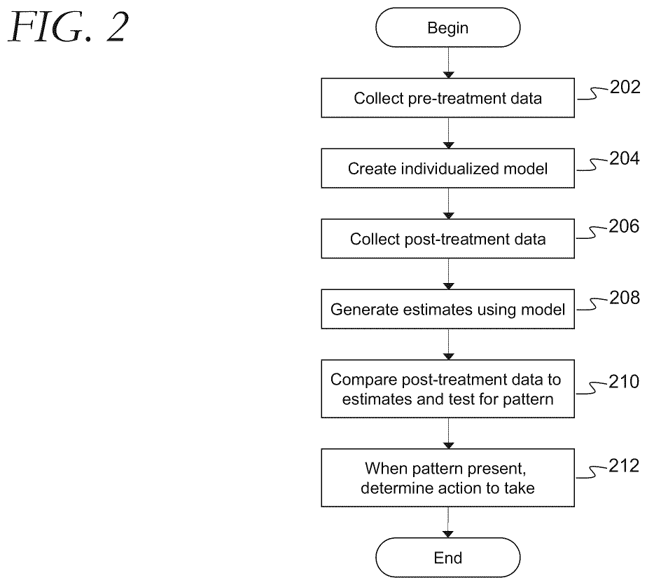

Referring now to FIG. 2, one example of an approach for detecting inflammatory responses in humans is described. At step 202, physiology data from at least one wearable sensor worn by the patient is collected during a pre-treatment interval.

At step 204, an individualized estimator model is created based on the patient's pre-treatment collected physiology data. The model is capable of estimating physiological variables responsive to receiving new physiology data from the at least one wearable sensor worn by the patient. The personalized/individualized estimator models are any multivariate model that provides as an output an estimate of the expected value of one or more vital sign values measured from the patient. Such models can be generated by Decision Tree/Random Forest function approximators; Similarity-based Models, and Neural Networks to mention a few examples.

At step 206, additional physiology data is collected from the at least one wearable sensor worn by the patient during a post-treatment interval. The sensors may be any type of sensor or sensor arrangement (including multiple sensors) that can be worn by humans and may be disposed at any convenient place on the human body such as the torso or wrist.

At step 208, estimates of the post-treatment physiology data using the individualized estimator model are generated. In aspects, the estimates are multiple and separate estimates for parameters such as heart rate, heart rate variability, respiration rate, activity, or temperature.

At step 210, post-treatment physiology data is compared to the estimates thereof and a determination is made as to when a pre-defined effect pattern is present based at least in part on the comparison. A difference may be taken as between each of the parameters or variables.

At step 212, when the predefined effect pattern is present, a determination of an action is made and the action is performed. The action is one or more of: triggering an electronic questionnaire to be prompted to the patient; providing instructions to the patient to take a measurement; providing instructions to the patient to contact the patient's clinician; triggering a ticket in a call center system to queue a call to the patient; creating a prompt in an app on the patient's phone to contact a clinician; providing instructions to the patient related to triaging the effects; transmitting a control signal to control medical equipment associated with treating the patient; and transmitting instructions to a vaccine manufacture to alter a composition and/or dosage of a vaccine. Other examples of actions are possible.

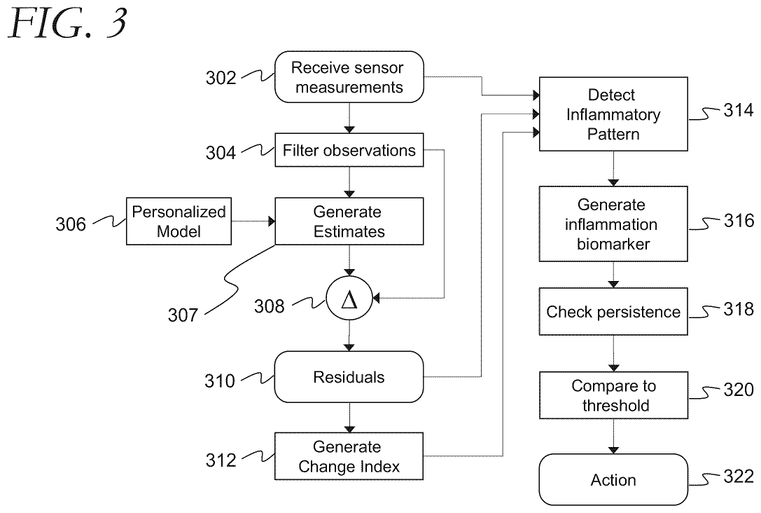

Referring now to FIG. 3, one overview of determining and utilizing residuals according to the approaches provided herein is described. It will be appreciated that the steps described with respect to FIG. 3 may be implemented as computer instructions executed on a processing device or control circuit. Vital signs values from sensor readings are received from a monitored patient at step 302. Multivariate observations of vital signs are filtered at step 304 to eliminate unacceptable or bad quality data or activity states that confound inflammation detection. Various criteria may be used to determine if the data is unacceptable.

An individualized estimator model 306 (trained as described elsewhere herein to be personalized to a particular human) receives the filtered data (having been filtered at step 304). As mentioned and described elsewhere herein, the personalized estimator models are any multivariate model that provides as an output an estimate of expected values of one or more vital sign values measured from a patient. Such models can be generated by Decision Tree/Random Forest function approximators; Similarity-based Models, and Neural Networks to mention a few examples.

The data (filtered at step 304) may comprise heart rate data, HRV data, respiration rate data, core temperature data, skin temperature data, and activity data obtained from sensors deployed to humans. Other examples are possible.

Once the data (filtered at step 304) is applied to the model 306, the model 306 responsively produces estimates at step 307. The estimates produced at step 307 represent what the model believes the data should be. In aspects, separate estimates are obtained for heart rate data, HRV data, respiration rate data, core temperature data, skin temperature data, and activity data.

A difference operator 308 takes the difference between the incoming data (filtered at step 304) and the estimates (produced at step 307) to produce the residuals 310. Residuals 310 encode how each of the vital signs as measured differ from what was expected based on pre-treatment data from the patient. In aspects, separate differences are obtained for the heart rate data, HRV data, respiration rate data, core temperature data, skin temperature data, and activity data.

Residuals 310 can also be combined into a single scalar value of overall change, a time series change index at step 312 (MCI as described elsewhere herein, or its alternates). A pattern representing the derangement of vital signs due to inflammation can then be applied to the residuals at step 314. The pattern in aspects is implemented as a compound rule set (e.g., and can be physically implemented as computer code or software), and can also utilize the Change Index as well as measured vital signs in rule evaluation, to augment the residuals 310. In aspects, the Change Index is useful for capturing overall change that is significant enough, while measured values may also play a role in confirming commonly understood indicia of inflammation (e.g., temperature). All residuals, change index and measured values are time series data, so that the application of the rule set to detect the inflammatory pattern also results in a time series, and represents an inflammation marker or biomarker at instant time points at step 316.

Given that inflammation may be evolving and may be inconsistently manifested in vital signs, it may become important to integrate (at step 318) the instant inflammation biomarker over time to produce a signal of persistent evidence of inflammatory response. Integration can be a time-windowed statistic or can be a process of latching (e.g., sometimes using or comparing to a threshold as described elsewhere herein) combined with area-under-the-curve integration and performed at step 320. The results determined at step 320 can be used to determine various actions at step 322. These actions are described elsewhere herein.

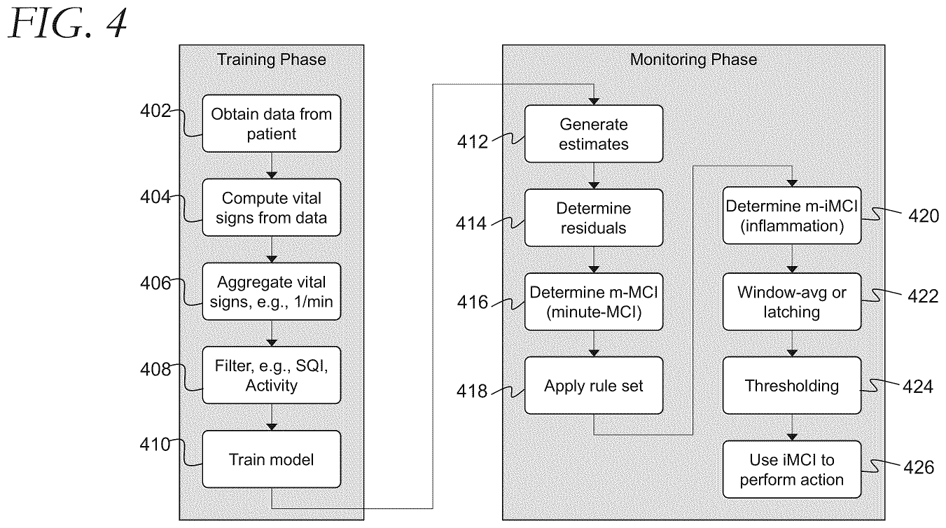

Referring now to FIG. 4, one example of an approach for determining inflammation effects and acting upon these effects is described. Steps 402-410 are part of the model training or learning process and the remaining steps part of the monitoring process (which occurs after the training or learning process has been completed).

At step 402, one or more sensors are placed on and worn by the patient and data is obtained. In some aspects, a torso-adherent sensor patch is used and continuously worn by the patient that enables measurement of several variables. These variables may include the heart rate and HRV from a single lead ECG, movement quantification (activity) from a 3-axis accelerometer, respiration rate from derivatives of movement, or amplitude/frequency modulation of HR, and/or skin temperature at the sensor site.

At step 404, vital signs can be computed from the waveform data for ECG and 3-axis accelerometer. By way of example, the ECG may be sampled at 125 Hz, and the accelerometer sampling rate may be 15 Hz or greater.

Heart rate may be computed on a beat-to-beat basis. Respiration rate may be computed at a 5-second sampling rate, though both higher and lower sampling rates are acceptable. Activity may be computed as a vector magnitude metric of movement (vibration) from all 3 axes, typically at 1 Hz, in any of a variety of units of measure that are known in the art. Temperature measurement is typically measured at 1 Hz, however is acceptable at lower sampling rates commensurate with expected rates of change with the human body or ambient conditions. In general, all variables should be measured at a rate of at least once per minute, and preferably at the higher rates described.

At step 406, vital signs (e.g., HR, RR, HRV, Activity, and Temperature) are then statistically summarized on a one-minute windowed basis. In aspects, this is a 10% trim mean of the values in the minute. But other statistics are acceptable and can be used, such as median, Nth percentile, mode, maximum or minimum.

At step 408, because of motion artifact and occasional loss of sufficient signal-to-noise ratio, a signal quality index (SQI) is used to identify when the vital signs are reliable for use in subsequent processing. In aspects, a signal quality index may be scaled from zero (e.g., indicating a bad or non-acceptable signal) to one (e.g., indicating a perfect signal) and can use the ECG waveform for assessment. Among methods for providing such a SQI, a deep neural network can be reliably trained to output a SQI that exhibits high correlation to human expert assessment of the usability of ECG traces. The SQI input may be a window of ECG 10-seconds long, containing a range of around 7 to around 30 heart beats (depending on heart rate), and may be assessed at a 5-second periodicity (e.g., 5-second overlap), by way of example. Other methods of producing a SQI are known to those skilled in the art using ECG waveforms. Given a SQI that ranges from 0 to 1, a threshold for excluding data for processing can be set to <0.8. Other examples are possible.

In addition to SQI, the level of activity may also serve as a filter for whether to use data or not for processing. An absolute value of activity level can be applied, above which the data will not be used. This provides a simple method of avoiding bad data and does not result in loss of much data for processing, as the level can be set such that a monitored human seldom exhibits that level of movement during the day. An activity threshold for excluding data from processing can be set at a level in units of movement that correspond to robust walking.

After the one-minute windowed means of the vital signs have been filtered using SQI and activity thresholding, what remains are samples of HR, RR, HRV, Activity and Temperature that can be collected from the monitored human and used to train a personalized baseline model.

At step 410 and as described elsewhere herein, a number of approaches can be used to create a baseline model (an individualized estimator model) that can generate estimates after training for comparison to measured values. In some aspects, a similarity-based model (SBM) is used. Such a model can be generated from as little as one or two diurnal cycles of continuous one-minute data from a human (1440 samples per day); however, more days of data can be used if the use case permits. For example, if a patient will receive a therapy at a later scheduled date, samples can be collected for a week to build a personalized model.

Once the model is trained, it enters or is utilized in a monitoring mode at step 412 where it generates estimates responsive to the input of each new sample of one-minute multivariate vital sign data obtained from the monitored individual. This happens continuously across all activities of daily living, subject to the aforementioned filters. As each multivariate reading of HR, RR, HRV, Activity and Temperature are obtained, they are input to the model to generate estimates of what the expected one-minute values for each are.

At step 414, residuals are generated by subtracting the estimates from the measured one-minute values. For example, where a one-minute heart rate is estimated to be lower than what is measured, the residual is positive, implying the actual heart rate of the human is higher than expected. Residuals are generated for each vital sign parameter, and residuals occur at the same sampling rate of once per minute.

At step 416, residuals are combined into a multivariate change index (MCI) that quantifies how much overall dispersion there is between the ensemble of measured vital signs and the ensemble of estimated vital signs. An MCI computation is described elsewhere herein. In aspects, MCI is scaled between 0 (meaning no anomalous behavior) and 1 (meaning high confidence of anomalous behavior). The MCI is also generated at a 1-minute sampling rate, with each input observation.

Using the available one-minute vital signs, residuals and MCI as inputs, a detection pattern specific for inflammation is applied in the form of a rule. At step 418, the rule outputs a modified MCI specific for inflammation, designated for purposes herein as iMCI. In one example, the rule logic follows these steps:
Initialize iMCI=0
If ((HRV_{RES}<=0) and (Temp_{RES}>=0) and (MCI>=0.1)) then:
If ((HR_{RES}>0.5) or (RR_{RES}>0.5) or (Temp_{RES}>0.2) or (HRV_{RES}<−0.025)) then:
Set iMCI=MCI
where:
HR_{RES} is measured in beats per minute.
RR_{RES} is measured in breaths per minute.
Temp_{RES} is measured in degrees Fahrenheit.
HRV_{RES} is measured in seconds.

The rule essentially zeroes out the MCI value unless the evidence is characteristic of inflammation, making the iMCI specific for inflammation. The first part of the rule provides gating against relatively higher HRV or relatively lower temperature or generally insufficient overall change (MCI). Put another way, the rule is fundamentally looking for sufficient derangement as quantified in the MCI, but only as long as that derangement is not due to higher-than-expected HRV or lower-than-expected temperature. Once this initial gating condition is met, the second part of the rule looks for any of possible positive evidence of inflammation in the form of higher-than-expected HR, or higher-than-expected RR, or higher-than-expected temperature or lower-than-expected HRV and determines a minute inflammation MCI (M_iMCI) at step 420. If any one or more of those forms of evidence are present at a level that surpasses their respective thresholds, then the iMCI is set to the value of the MCI.

The M_iMCI is a time series of values also at one minute sampling rate. At step 422 a check for persistence of the evidence of inflammatory physiology derangement is made by summing or averaging the iMCI values over a window, which also serves to smooth the signal from transient noise. In some aspects, this window is 3 hours, and the average evaluated every 15 minutes (2-hour 45-minute overlap). Other statistical characterization of the windowed iMCI are possible.

In other aspects and at step 422, the one-minute iMCI time series is “latched” to the highest value, when time series values persistently exceed a “latching” threshold until a sufficiently lower sequence of values drop below a threshold, at which point the iMCI is unlatched from the higher value and remains un-latched at the lower values. Then the 3-hour summation or average of iMCI is made on the latched version of iMCI at step 424.

Latching can be understood with an integer toy example: Given a time series of instant values as shown in the first series below, a latching threshold of 6, and persistence requirement of two samples, The sequence is latched to the highest value after the first sequence of 6 and 7 until the original sequence falls below a value of 6 for at least two samples in a row at which point the samples are un-latched, as shown in the second series below:
[4, 6, 5, 5, 6, 7, 8, 7, 6, 7, 5, 3, 1, 2, 1]
[4, 6, 5, 5, 6, 7, 8, 8, 8, 8, 8, 8, 1, 2, 1]

By itself, one-minute iMCI or the 3-hour windowed (summed or averaged) iMCI or latched-iMCI with 15-minute sample rate serves as a quantifier of suspected inflammatory response. This can be useful in quantifying trends or assessing change from before to after a treatment in a drug trial. A drug study in patients can examine the change in windowed iMCI/latched-iMCI from before to after treatment administration, and compare responses of different cohorts, e.g., Treatment Group vs Control Group. Any of one-minute iMCI, one-minute latched iMCI, windowed iMCI or windowed latched-iMCI can be tallied on a unit time basis (e.g., daily) to quantify overall relative inflammatory response present in the physiology of any patient or trial participant.

Actions can be taken at step 426. In the case where it is useful to detect and escalate notification of inflammation, e.g., acute inflammatory episode or cytokine release syndrome, windowed iMCI/latched-iMCI can also be compared to a threshold, and if the value exceeds the threshold, an acute level of inflammation is recognized, and an alert is provided to clinicians and/or to the patient to take measures to mitigate the acute response. In some aspects, a notification can be triggered where the value of the windowed, latched iMCI exceeds 0.25. This threshold serves to distinguish an acute level of inflammation (which requires intervention) from an expected level of inflammation (which does not require triage). This can be useful for example where a cancer treatment is being used that harnesses the power of the human immune system. There is an expectation of a moderate level of inflammation, which occurs because the treatment is working against the cancer, but a risk of an acute episode of inflammation, e.g., a cytokine “storm” than can be deadly.

In still other aspects, the threshold used to escalate or send an alert for acute inflammation is applied to the sample-over-sample difference in the windowed iMCI/latched-iMCI. In other words, successive 15-minute samples of 3-hour windowed values are checked for an abrupt rise. If a rise at least as large as the threshold is seen, regardless of absolute value, the alert is triggered.

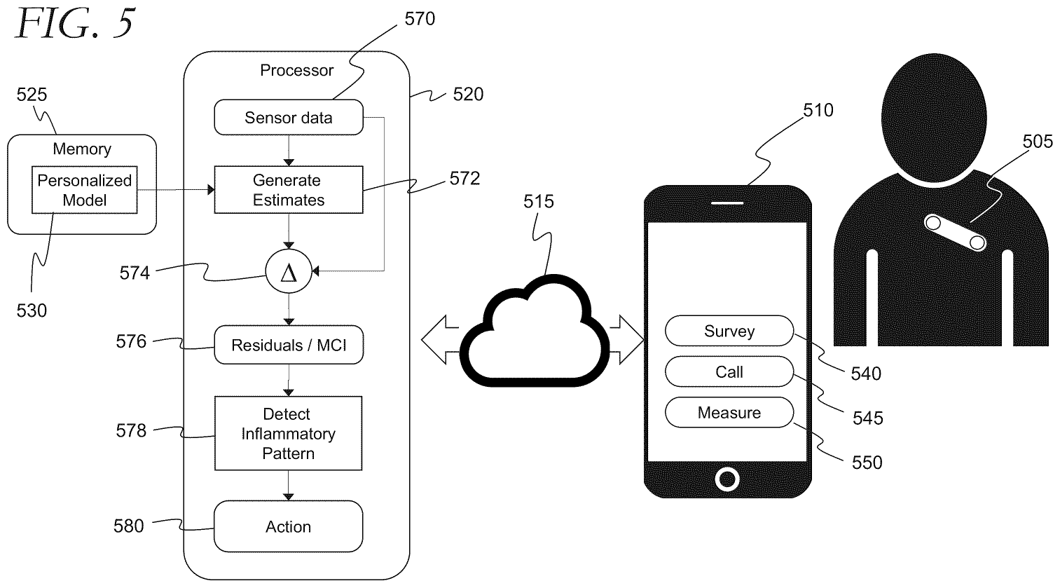

Turning to FIG. 5, a safety monitoring system is illustrated for a patient receiving a medical treatment (e.g., for a patient that may have undesired cytokine release syndrome as a side-effect, which may be dangerous to the patient). In aspects, this system provides a safety net for the patient after leaving the acute care setting and moving to the home environment, which is remote from the acute care facility. The system comprises a wearable torso patch sensor 505 that transmits data via Bluetooth radio (or some other communication technology or protocol) to a smartphone 510 in the possession of the patient. Sensor data is transmitted by an app (e.g., a software application) on the smartphone over public data infrastructure 515, which may be the internet or other mobile data infrastructure, to a data center (also called a central call center) containing processor unit (or control circuit) 520 connected to data storage memory 525. The central call center or data center may be or may be associated with a doctor's office, hospital, acute care facility, group of hospitals, clinics, any health care facility or facilities, or may be a data processing center located at a geographic location that is different from health care facilities. Other locations are possible. The processor 520 and associated data storage memory 525 may be physically located at the central call center but also may be located at other locations such as the cloud or at another health care facility.

The patient's prior pre-treatment sensor data has been used to train a personalized multivariate estimator model 530 stored in the memory 525 and accessible for computing steps by processor 520, which steps include generating estimates of expected values for data measured by the sensor 505, and comparing them to generate residuals, and univariate scores like MCI. Residuals and MCI are in turn used in detection signatures for escalating inflammation processes in the patient.

Buttons may be used on the smartphone 510 to instigate actions. When a button push, press, or actuation is detected, processor 520 transmits instructions to the app on the smartphone to prompt one or more actions to the patient embodied in the on-screen button features: A button 540 to fill out a survey on the smartphone which can confirm or deny symptoms related to inflammation; a button 545 that automatically puts the patient in contact with a clinician responsible for managing the patient, which may include a call center of trained personnel with those duties; and a button 550 to make and report additional measurements using instruments the patient has been provided with at home, such as a thermometer for taking core body temperature, a blood pressure measurement device and/or a pulse oximeter. In aspects and as mentioned, the buttons 540, 545, and 550 may be displayed on the screen of the smartphone 510. A user may press or otherwise actuate these buttons by touching and pressing the screen at the appropriate area where the button is displayed (e.g., using their fingers, a cursor, or a stylus). Alternatively, the buttons 540, 545, and 550 may be physical buttons on the smartphone 510 that are physically depressed by a user.

More specifically and during some operations, the processor 520 takes sensor data 570, applies this to generate estimates at step 572, uses a difference operator 574 to take the difference between the sensor data 570 and estimates (obtained at step 572) to obtain residuals and then determine MCI as described herein at step 576. Rules can be used to determine an inflammatory pattern at step 578 and an action taken at step 580. These steps are described in greater detail elsewhere herein.

Regarding button 540, for example, pressing the button causes the display of a survey on the screen of the smartphone 510. The survey may include several pages (each page being on a separate screen) that are paged through by the patient. As the patient pages through the survey, they can answer the questions. In one example, they may type in information and/or tick boxes depending upon the nature and format of the survey. In another example, the patient may enter information verbally into the smartphone 510. That is, once a question is presented the answer may be spoken by the patient, a microphone on the smartphone 510 may receive spoken information, and the information may be received by a processor or control circuit of the smartphone for further processing.

Regarding button 545, for example, the patient may be identified by a patient number, code, and/or other suitable identifier. Once the button 545 is pushed, then the app transmits an electronic message that includes the code to a call center (or other facility). In these regards, pressing the button 545 may establish a telephone, video, and/or other type of electronic communication connection or link with the central call center. Each patient (via their patient number or code) may be associated with a particular clinician. Thus, and in one example, the control circuit 520 at the central call center (associated with the clinicians) may receive the patient code and/or number, identify the clinician associated with that number or code, and then establish the link between the patient and the associated clinician.

The central call center may, for each separate clinician, have separate and different procedures stored to reach or contact different clinicians. Each of these may be stored as separate programs or computer instructions (e.g., stored at the memory 525) that are executed as needed (e.g., by the control circuit 520). For example, for a first clinician, a text message is automatically generated instructing the clinician to call the patient. In another example, a call (or other electronic communication) is automatically made to the clinician and when the clinician affirms, a telephone link or connection is established directly between the clinician and the patient. It will be appreciated that execution of such computer instructions automatically controls electronic communication equipment by setting switches, routing messages across various networks and components of these networks such as gateways and otherwise controls various electronic devices, for example, within the infrastructure 515. Once the appropriate clinician is identified, the control circuit 520 at the central call center may identify, retrieve, and then execute the appropriate program to contact the clinician.

Security or verification procedures may also be automatically and/or manually performed at the central call center to verify the patient's identity. For example, a patient's code may be checked against a database of valid patient codes by the control circuit 520. In other aspects, security questions may be presented to the patient on the smartphone before the process can continue. In other words, the patient's correct response to these questions may be required to continue with the process.

More specifically, the patient's answers may be sent to the central call center and the control circuit 520 at the central call center may compare the patient's answers to answers stored at the central call center (e.g., at the memory 525). Correct matches allow the link to be established and/or information to be exchanged. Incorrect matches may halt processing operations with respect to the patient. In these regards, any programs attempting to set up a link may be halted and/or an electronic message set back to the smartphone 510 informing the patient that they have not passed the security check.

In yet other examples, biometric information may indicate the identity of a patient. In aspects, the patient may place their finger on the screen of the smartphone 510 (or some other device that obtains the fingerprint that is connected to the smartphone 510), and this information is sent to the central call center. The control circuit 520 compares the fingerprint to a stored fingerprint on file at the central call center.

In still other examples, the instigation of this process may cause retrieval of insurance information of the patient. The insurance information may be stored, in some examples, at the central call center, but in other examples may be stored at an external source. If required, another electronic connection may be established (e.g., automatically by the control circuit 520) between the central call center and the external source to access, retrieve, and/or inquire about the insurance information. Insurance information may be used to determine whether to proceed with the process or how to proceed. In some examples, depending upon the nature of the insurance information the process and any programs executing that are implementing the process may be electronically halted by the control circuit 520 at the central call center. Infrastructure 515 may be used to establish any connections needed to obtain information from external sources.

Regarding button 550, the reporting may be manually typed in or may be captured from the measurement device by Bluetooth by the app on the smartphone. In some aspects, the patient may first establish communication links between the smartphone 510 and the additional instruments. For example, the smartphone, in a setup mode, may attempt to detect the presence of the additional device. This can be accomplished using a number of different protocols where, once detected, the link is established. For example, the smartphone 510 may send a signal to the additional device, and the additional device may respond. Alternatively, the additional device may broadcast a signal that is detected by the smartphone 510. Once a link is established, the smartphone 510 may receive information periodically or continuously from the additional device.

Measurements made by the patient or survey answers provided by the patient are automatically uploaded by the app to the data center which notifies clinicians of the detected inflammation pattern and the information provided by the patient responsive thereto. Using this system has the enormous advantages of providing continuous monitoring of the patient in the at-home setting without the cost of full-time admission to an acute care facility. Furthermore, the personalized model-base detection of early warning signs of inflammation escalation coupled with the automated actions pushed to the patient provide a more actionable context for decision making by the clinician to whom any detection is escalated, improving timeliness of intervention if warranted.

The approaches provided herein are advantageous for a variety of different reasons. For example, the approaches are individualized and provide better sensitivity and specificity. These approaches learn from data how a person's physiology behaves, so that when inflammation has an impact on that physiology, the change stands out.

In addition, the approaches provided herein are objective. They do not rely on subjective perceptions, as would symptom reports.

Further, the approaches provided herein requires no special reliance on the patient to proactively perform tasks to collect data. The patient only has to wear a sensor. In contrast, other methods of trying to detect severe inflammatory responses include asking the patient to take their temperature periodically. A reliance on the patient to do something at intervals is always problematic.

The present approaches use an individualized model tuned to the specific patient. The present approaches also look for evidence of the adverse side effects as patterns in the differences between what the model estimates and what was actually measured from the sensor on the patient.

The present approaches provide instructions to a patient to address the side effects. These instructions could be instructions to fill out a questionnaire with symptom confirmation or severity, instructions to come into clinic for testing or treatment, instruction to take an ameliorative drug, and/or instructions to make a manual measurement with another device.

The model can be characterized either (a) as “dynamic,” such that input data to the model plays a role in how the estimates come out, or (b) as a “multivariate” model. The “estimate” is the static average; the difference from that static value is deemed a “personalized” residual that can be tested for the “signature” (large difference) of inflammation.

Those skilled in the art will recognize that a wide variety of other modifications, alterations, and combinations can also be made with respect to the above-described embodiments without departing from the scope of the invention, and that such modifications, alterations, and combinations are to be viewed as being within the ambit of the inventive concept.

What is claimed is:

1. A method for monitoring a patient for effects of a pharmacological therapy comprising the steps of:
collecting physiology data from at least one wearable sensor worn by the patient during a pre-treatment interval;
creating an individualized estimator model based on the patient's pre-treatment collected physiology data, the model capable of estimating physiological variables responsive to receiving new physiology data from the at least one wearable sensor worn by the patient;
collecting additional physiology data from the at least one wearable sensor worn by the patient during a post-treatment interval;
generating estimates of the post-treatment physiology data using the individualized estimator model;
comparing post-treatment physiology data to the estimates thereof and determining when a pre-defined effect pattern is present based at least in part on the comparison; and
when the predefined effect pattern is present, determining and performing an action, the action being one or more of:
triggering an electronic questionnaire to be prompted to the patient;
providing instructions to the patient to take a measurement;
providing instructions to the patient to contact the patient's clinician;
triggering a ticket in a call center system to queue a call to the patient;
creating a prompt in an app on the patient's phone to contact a clinician;
providing instructions to the patient related to triaging the effects;
transmitting a control signal to control medical equipment associated with treating the patient;
transmitting instructions to a vaccine manufacture to alter a composition and/or dosage of a vaccine.

2. The method of claim 1, wherein the effects are adverse side effects.

3. The method of claim 1, wherein the effects are signs of efficacy of a vaccine.

4. The method of claim 1, wherein the physiology data comprises heart rate data, respiration rate data, core temperature data, skin temperature data, and activity data.

5. The method of claim 1, wherein the individualized estimator model is trained using data collected from the patient while in a free-living physiological state where an inflammatory status of the patient is stable and not expected to be changing.

6. The method of claim 1, wherein comparing comprises determining residuals between the estimates and the physiology data.

7. The method of claim 6, wherein the residuals are synthesized into a singular score, the score being a scalar index.

8. The method of claim 1, wherein the individualized estimator model comprises a neural network.

9. A system for monitoring a patient for effects of a pharmacological therapy, the system comprising:
at least one wearable sensor worn by a patient during a pre-treatment interval, the at least one wearable sensor configured to collect physiology data from the patient;
a control circuit coupled to the at least one wearable sensor, the control circuit configured to:
create an individualized estimator model based on the patient's pre-treatment collected physiology data, the model capable of estimating physiological variables responsive to receiving new physiology data from the at least one wearable sensor worn by the patient;
wherein the at least one wearable sensor collects additional physiology data from the at least one wearable sensor worn by the patient during a post-treatment interval;
wherein the control circuit is further configured to:
generate estimates of the post-treatment physiology data using the individualized estimator model;
compare post-treatment physiology data to the estimates thereof and determine when a pre-defined effect pattern is present based at least in part on the comparison; and
when the predefined effect pattern is present, determine and perform an action, the action being one or more of:
provide instructions to the patient related to triaging the effects;
transmit a control signal to control medical equipment associated with treating the patient;
transmit instructions to a vaccine manufacture to alter a composition and/or dosage of a vaccine.

10. The system of claim 9, wherein the effects are adverse side effects.

11. The system of claim 9, wherein the effects are signs of efficacy of a vaccine.

12. The system of claim 9, wherein the physiology data comprises heart rate data, respiration rate data, core temperature data, skin temperature data, and activity data.

13. The system of claim 9, wherein the control circuit trains the individualized estimator model using data collected from the patient while in a free-living physiological state where an inflammatory status of the patient is stable and not expected to be changing.

14. The system of claim 9, wherein the control circuit is configured to compare by determining residuals between the estimates and the physiology data.

15. The system of claim 14, wherein the control circuit is configured to synthesize the residuals into a singular score, the score being a scalar index.

16. The system of claim 9, wherein the individual estimator model comprises a neural network.
